(19) United States
(12) Patent Application Publication
(10) Pub. No.: US 2026/0366086 A1
(43) Pub. Date: Nov. 19, 2026

(54) SNP molecular marker associated with quality traits in goose meat

(71) Applicant: CHONGQING ACADEMY OF ANIMAL SCIENCES, Chongqing (CN)

(72) Inventors: Guangliang GAO, Chongqing (CN); Qigui WANG, Chongqing (CN); Ping HUANG, Chongqing (CN); Xianzhi ZHAO, Chongqing (CN); Keshan ZHANG, Chongqing (CN); Zhuping CHEN, Chongqing (CN); Yi LUO, Chongqing (CN); Youhui XIE, Chongqing (CN); Ying CHEN, Chongqing (CN); Xiaofeng HUANG, Chongqing (CN); Jiajia XUE, Chongqing (CN); Zuolan LIU, Chongqing (CN); Wengui FU, Chongqing (CN); Guoyang XU, Chongqing (CN); Chao WANG, Chongqing (CN); Zhen WANG, Chongqing (CN)

(73) Assignee: CHONGQING ACADEMY OF ANIMAL SCIENCES, Chongqing (CN)

(21) Appl. No.: 18/841,385

(22) PCT Filed: Sep. 15, 2023
(86) PCT No.: PCT/CN2023/118964

§ 371 Date: Jul. 13, 2026

(30) Foreign Application Priority Data
Jun. 15, 2023 (CN) 2023107071007

(51) Int. Cl. C12Q 1/6888

(52) U.S. Cl. CPC C12Q 1/6888; C12Q 2600/13; C12Q 2600/156

(57) ABSTRACT

The present invention discloses a SNP molecular marker associated with quality traits in goose meat and relates to the field of gene sequencing. The technical solution provided by the present invention is aimed at screening and identifying single nucleotide polymorphisms (SNPs) associated with quality traits in goose meat and a genome-wide association analysis (GWAS) was performed to screen SNPs in the goose genome of 70-day-old male Anser cygnoides domesticus. In addition, genotypes of selected SNPs associated with goose meat were detected by the matrix-assisted laser desorption ionization time-of-flight mass spectrometry (MALDITOP MS) method, which showed that 43 SNPs were significantly associated with these traits. Furthermore, 28 annotated genes were detected as potential candidates associated with five meat quality traits. A gene enrichment analysis in the 1 Mb range near SNPs showed significant enrichment in pathways of protein digestion and absorption as well as glycerolipid metabolism.

### TECHNICAL FIELD

The present invention relates to the field of gene sequencing, and more particularly to a SNP molecular marker associated with quality traits in goose meat.

### BACKGROUND TECHNOLOGY

As a nutritious meat, goose meat is considered as a healthy food due to high protein, low fat and low cholesterol, and is regarded as one of the ideal meat products by countries around the world, which has become an important source of meat food. According to the WHO in 2021, the goose meat production in China reached an alarming 570 million, accounting for 99% of total production in Asia and 94% of total global production. In addition, China's goose meat production reached 1.77 million tons. The Anser cygnoides domesticus, a unique local breed in China, is known for its excellent meat quality, enjoys a reputation and possesses great market values for its soft and delicate texture. The demand for goose meat has risen steadily in recent years. This surge can be attributed to a growing concern for healthy lifestyles, highlighting significant opportunities for the goose industry. Goose meat quality is a multifaceted attribute shaped by a multitude of genes, including both primary and secondary influences. The complexity and multifaceted regulatory mechanisms that govern the attribute make the meat quality even more complicated. Previous studies have extensively documented the effects of factors such as breed, age, nutrition, environment and disease on quality traits in goose meat. Therefore, there is great potential to identify and screen molecular markers and key genes associated with these traits, which can improve breeding efficiency and facilitate the overall quality of goose meat. Meat quality evaluation relies on a plurality of meat indicators such as brightness (L*), redness (a*), yellowness (b*), pH, shear force (SF), cooking loss rate (CLR), and roughness. Methionine-rich diets have been shown to positively affect pectoral muscle production, increase pH, enhance meat color, and reduce muscle shear, with the presence of fatty acids in the diet affecting meat flavor and intramuscular fat production directly. The studies have shown that higher levels of soy lecithin in the diet increase the fat content of pectoral and leg muscles, which improves binding, reduces drip loss, and minimizes cooking loss in the muscle. In addition, an increase in crude fat content (CFC) promotes intermuscular fat deposition and weakens the bonding between muscle fibers, resulting in a reduction in shear force and an increase in meat tenderness.

Genome-wide association studies (GWAS) investigating relationships between population phenotypes and genotypes have made important discoveries in identifying genetic variation associated with meat quality traits in livestock and poultry. Advances in whole genome resequencing have allowed accurate screening of various types of genetic variants, including single nucleotide polymorphisms (SNPs), InDels, structural variants (SVs), copy number variants (CNVs), and presence/absence variants (PAVs). However, no single nucleotide polymorphisms (SNPs) associated with meat quality traits have been successfully detected in the genome of Anser cygnoides domesticus by the whole genome resequencing.

### SUMMARY OF THE INVENTION

In order to solve the above technical problems, the present invention provides a SNP molecular marker associated with quality traits in goose meat, performs a GWAS analysis of quality traits in goose meat, and identifies 43 SNPs significantly associated with these traits, which helps to comprehensively understand the genetic mechanism of quality traits in goose meat.

The technical solution adopted by the present invention to solve the technical problem is: a SNP molecular marker associated with quality traits in goose meat, wherein the SNP molecular marker is as follows:

| SNP | Chr | Position (bp) | allele1 | Traits | P value | Gene |
|---|---|---|---|---|---|---|
| chr11: 9193757 | chr11 | 9193757 | G/A | CFC | 8.12 × 10⁻⁰⁸ | TMEM65 |
| chr19: 2113530 | chr19 | 2113530 | C/T | CFC | 6.34 × 10⁻⁰⁸ | SMAD6 |
| chr19: 17076992 | chr19 | 17076992 | T/G | CLR | 1.03 × 10⁻⁰⁷ | SYTC2 |
| chr27: 11913710 | chr27 | 11913710 | G/A | CLR | 3.16 × 10⁻⁰⁸ | MSI1H |
| chr1: 42733613 | chr1 | 42733613 | T/C | L*(meat lightness) | 7.56 × 10⁻⁰⁸ | LIN1 |
| chr14: 440052 | chr14 | 440052 | T/C | L*(meat lightness) | 8.15 × 10⁻⁰⁸ | GSS |
| chr1: 1971942 | chr1 | 1971942 | C/T | b*(meat yellowness) | 2.74 × 10⁻⁰⁸ | EXTL3 |
| chr1: 24867798 | chr1 | 24867798 | C/G | b*(meat yellowness) | 7.82 × 10⁻⁰⁸ | PRSS55 |
| chr1: 31137893 | chr1 | 31137893 | C/T | b*(meat yellowness) | 9.31 × 10⁻⁰⁸ | PTP4A1 |
| chr3: 31387295 | chr3 | 31387295 | G/A | b*(meat yellowness) | 5.43 × 10⁻⁰⁹ | TMEM19 |
| chr3: 31387314 | chr3 | 31387314 | G/A | b*(meat yellowness) | 3.57 × 10⁻⁰⁸ | TMEM19 |
| chr3: 31387324 | chr3 | 31387324 | A/G | b*(meat yellowness) | 3.57 × 10⁻⁰⁸ | TMEM19 |
| chr7: 8983345 | chr7 | 8983345 | A/G | b*(meat yellowness) | 3.20 × 10⁻⁰⁸ | ACKR3 |
| chr7: 16992800 | chr7 | 16992800 | C/T | b*(meat yellowness) | 7.84 × 10⁻⁰⁸ | CHIN |
| chr7: 4199190 | chr7 | 4199190 | A/G | b*(meat yellowness) | 4.47 × 10⁻⁰⁸ | GULP1 |
| chr9: 33911228 | chr9 | 33911228 | A/G | b*(meat yellowness) | 7.35 × 10⁻⁰⁸ | DOCK1 |
| chr11: 27962909 | chr11 | 27962909 | A/G | b*(meat yellowness) | 3.43 × 10⁻⁰⁸ | PP4R1 |
| chr11: 27964588 | chr11 | 27964588 | G/T | b*(meat yellowness) | 4.66 × 10⁻⁰⁹ | SLCO5A1 |
| chr13: 25432644 | chr13 | 25432644 | C/T | b*(meat yellowness) | 2.55 × 10⁻⁰⁹ | CATC |
| chr13: 25426268 | chr13 | 25426268 | G/A | b*(meat yellowness) | 2.62 × 10⁻⁰⁹ | CATC |
| chr13: 25389761 | chr13 | 25389761 | T/G | b*(meat yellowness) | 9.87 × 10⁻⁰⁸ | CATC |
| chr26: 4307814 | chr26 | 4307814 | T/C | b*(meat yellowness) | 4.26 × 10⁻⁰⁸ | PP4R1 |
| chr29: 8720675 | chr29 | 8720675 | T/C | b*(meat yellowness) | 1.16 × 10⁻⁰⁸ | MRPS2 |
| chr33: 5370910 | chr33 | 5370910 | A/C | b*(meat yellowness) | 6.90 × 10⁻⁰⁸ | NO40 |
| chr33: 5659799 | chr33 | 5659799 | A/C | b*(meat yellowness) | 3.33 × 10⁻⁰⁸ | PUM1 |
| chr33: 3667950 | chr33 | 3667950 | C/T | b*(meat yellowness) | 8.99 × 10⁻⁰⁸ | RRAGC |
| chr36: 5611038 | chr36 | 5611038 | G/T | b*(meat yellowness) | 4.99 × 10⁻⁰⁸ | GOGA7 |
| ctg2092: 176333 | ctg2092 | 176333 | C/T | b*(meat yellowness) | 6.16 × 10⁻⁰⁸ | RXRA |
| ctg745: 127150 | ctg745 | 127150 | A/G | b*(meat yellowness) | 5.53 × 10⁻⁰⁸ | NCOA2 |
| chr4: 23787309 | chr4 | 23787309 | A/G | b*(meat yellowness) | 2.08 × 10⁻⁰⁸ | None |
| chr4: 49863664 | chr4 | 49863664 | T/A | b*(meat yellowness) | 5.49 × 10⁻⁰⁸ | None |
| chr25: 12875901 | chr25 | 12875901 | A/G | b*(meat yellowness) | 5.77 × 10⁻⁰⁹ | None |
| chr29: 8278595 | chr29 | 8278595 | C/T | b*(meat yellowness) | 6.64 × 10⁻⁰⁸ | None |
| ctg834: 24329 | ctg834 | 24329 | C/T | b*(meat yellowness) | 5.15 × 10⁻⁰⁸ | None |
| ctg930: 11921 | ctg930 | 11921 | G/A | b*(meat yellowness) | 5.30 × 10⁻⁰⁹ | None |
| ctg956: 92804 | ctg956 | 92804 | C/T | b*(meat yellowness) | 2.73 × 10⁻⁰⁸ | None |
| chr2: 36812553 | chr2 | 36812553 | C/T | SF (kgf) | 5.94 × 10⁻⁰⁸ | BMF |
| chr2: 36812412 | chr2 | 36812412 | G/C | SF (kgf) | 9.22 × 10⁻⁰⁸ | BMF |
| chr7: 33558795 | chr7 | 33558795 | A/T | SF (kgf) | 3.84 × 10⁻⁰⁸ | NEMP2 |
| chr7: 33558758 | chr7 | 33558758 | C/A | SF (kgf) | 8.04 × 10⁻⁰⁸ | NEMP2 |
| chr7: 33558832 | chr7 | 33558832 | T/C | SF (kgf) | 9.30 × 10⁻⁰⁸ | NEMP2 |
| chr12: 31782870 | chr12 | 31782870 | A/G | SF (kgf) | 2.78 × 10⁻¹⁰ | NAD-ME |
| chr12: 31781825 | chr12 | 31781825 | G/A | SF (kgf) | 3.62 × 10⁻⁰⁹ | NAD-ME |

Preferably, 11 SNPs genotypes include:
genotypes associated with b* meat yellowness:
chr13:25432644;
chr13:25432644;
ctg930:11921;
chr36:5611038;
ctg2092:176333;
chr29:8278595;
chr9:33911228;
chr1:24867798;
chr7:16992800;
genotypes associated with SF meat:
chr12:31782870;
chr12:31781825;
chr2:36812553.

Preferably, primer sequences of MALDI-TOF MS are as follows:

| SNPs | 2nd-PCRP | 2nd-PCRP | UEP_SEQ |
|---|---|---|---|
| chr13: | ACGTTGGATGGAAAT | ACGTTGGATGACCTG | agaaCCTGTTGTCTC |
| 25432644 | ACCCTGTTGTCTCCC | TTTGACTCCTTTTGG | CCTCACTC |
| ctg930: | ACGTTGGATGTGCCA | ACGTTGGATGTGGCA | GCCCCCTCCTGCACC |
| 11921 | CCGCAGGGATCACG | GCAGGGTGGGGAAA | TT |
| chr36: | ACGTTGGATGTTCCC | ACGTTGGATGCCCAG | tCGCTTTGATTTAGT |
| 5611038 | CCCTCGTTTGAATTG | TCTGAATTCCAACAC | TATTTTACTC |
| ctg2092: | ACGTTGGATGGGAAT | ACGTTGGATGTTTTG | cccaTAGACTACGTC |
| 176333 | ATGTAGACTACGTCT | GACAAACAGGAGACC | TGCCATCT |
| | G | | |
| chr29: | ACGTTGGATGAAGGA | ACGTTGGATGTGCAC | CAGGAACCGAGGGAA |
| 8278595 | TTTGGGAAGCAGGAA | CGGGGAGGAGGAGA | ATGC |
| | C | | |
| chr9: | ACGTTGGATGCCTGG | ACGTTGGATGCTTAA | CAGGAGTTAAGGGAG |
| 33911228 | AGGCAATCAAAGATG | GTCGCCTTGGTACAC | AAAAT |
| chr1: | ACGTTGGATGAGCCT | ACGTTGGATGGTGTG | GCTGCCTGCCCAGAA |
| 24867798 | GATGCAGTCACATCC | TGCCAGACAAAACTC | CT |
| chr7: | ACGTTGGATGTCACA | ACGTTGGATGTTATA | gtCAGGGTCCAACTC |
| 16992800 | TTGGCAGGGTCCAAC | GTCTGCTCTGGACTG | AGTCTCC |
| chr12: | ACGTTGGATGACAAA | ACGTTGGATGGGCAG | CAATAATGTTTAACG |
| 31782870 | GAACACATCGCAAGG | CAGCTTTCAGCAAAC | TTAGACTC |
| chr12: | ACGTTGGATGTTCTG | ACGTTGGATGTTTAA | CCAAAGACCTGTTGA |
| 31781825 | CAACGCTCGAAATCC | CCTACGCATGCCTCC | GA |
| chr2: | ACGTTGGATGGTGGA | ACGTTGGATGGCAGG | tcGATCTTACTTTTT |
| 36812553 | AGAAGACATCACTGG | AGAAAAAAGCATAAG | TATCTTCCATTA |

Preferably, frequencies and genotypes of selected SNPs associated with goose meat quality are as follows:

| Traits | Number | SNPs | Genotypes (Frequency %) | | |
|---|---|---|---|---|---|
| b*(meat yellowness) | 215 | chr13:25432644 | CC | CT | TT |
| | | | 17.61 ± 2.07^{b} (1.97) | 17.56 ± 2.92^{b} (0.66) | 19.31 ± 0.25^{a} (97.37) |
| b*(meat yellowness) | 215 | ctg930:11921 | AA | GA | GG |
| | | | 19.18 ± 0.23^{b} (72.37) | 20.28 ± 0.41^{a} (23.03) | 19.03 ± 0.88^{b} (4.61) |
| b*(meat yellowness) | 215 | chr36:5611038 | GG | GT | TT |
| | | | 19.83 ± 2.76^{a} (0.58) | 17.71 ± 1.04^{b} (4.62) | 19.44 ± 0.22^{a} (94.80) |
| b*(meat yellowness) | 215 | ctg2092:176333 | CC | TC | TT |
| | | | 18.04 ± 1.15^{b} (3.49) | 19.24 ± 0.45^{a} (13.56) | 19.48 ± 0.26^{a} (70.93) |
| b*(meat yellowness) | 215 | chr29:8278595 | CC | CT | TT |
| | | | 17.99 ± 2.80^{b} (0.56) | 19.21 ± 0.60^{a} (13.56) | 19.39 ± 0.23^{a} (85.88) |
| b*(meat yellowness) | 215 | chr9:33911228 | AA | GA | GG |
| | | | 20.32 ± 1.62^{a} (2.27) | 19.52 ± 0.75^{b} (8.52) | 19.31 ± 0.23^{b} (89.20) |
| b*(meat yellowness) | 215 | chr1:24867798 | CC | GC | GG |
| | | | 16.33 ± 2.79^{b} (0.57) | 19.08 ± 0.72^{a} (9.09) | 19.41 ± 0.23^{a} (90.34) |
| b*(meat yellowness) | 215 | chr7:16992800 | CC | CT | TT |
| | | | 15.43 ± 2.77^{b} (1.12) | 19.30 ± 0.57^{a} (13.48) | 19.40 ± 0.23^{a} (85.39) |
| SF | 215 | chr12:31782870 | AA | GA | GG |
| | | | 3.12 ± 0.97^{b} (1.12) | 3.82 ± 0.25^{a} (8.43) | 3.74 ± 0.08^{a} (90.45) |
| SF | 215 | chr12:31781825 | AA | GA | GG |
| | | | 3.72 ± 0.08^{b} (87.01) | 3.89 ± 0.22^{a} (12.99) | \ |
| SF | 215 | chr2:36812553 | CC | CT | TT |
| | | | \ | 3.60 ± 0.22^{b} (15.49) | 3.73 ± 0.09^{a} (84.51) |

Beneficial effects of the present invention:

The present invention provides the GWAS analysis on quality traits in goose meat and identified 43 SNPs significantly associated with these traits, which helps to fully understand the genetic mechanism of quality traits in goose meat and lay a solid foundation for future research in this field.

### BRIEF DESCRIPTION OF THE DRAWINGS

The technical solution provided by the embodiments of the present invention or prior art will be clearly and completely understood from the following brief description by reference to the drawings. It will be apparent that the accompanying drawings in the following description are only three of the drawings of the present invention, and that other drawings may be obtained from these drawings without creative labor for those of ordinary skill in the art.

FIG. 1 illustrates a schematic diagram of SNP density within a 1 Mb window size of a goose genome according to an embodiment of the present invention;

FIG. 2 and FIG. 3 illustrate Manhattan and Q-Q plots of different meat quality traits of male Anser cygnoides domesticus according to an embodiment of the present invention. Note: The figure shows the following traits: CFC (A, B); CLR (C, D); L* (meat brightness) (E, F); b* (meat yellowness) (G, H); SF (I, J), where the significance threshold is set at 1×10⁻⁷;

FIG. 4 illustrates a schematic diagram of a functional analysis of genes up to 1 Mb in SNPs associated with quality traits in goose meat according to an embodiment of the present invention.

### SPECIFIC EMBODIMENTS

The present invention will be explained below in more detail by reference to drawings and embodiments. However, the embodiments are given for clearness of understanding only and no unnecessary limitations should be understood therefrom.

### Embodiments

### 1 Materials and Methods

### 1.1 Laboratory Animals and Phenotypic Traits

Male Anser cygnoides domesticus as experimental animals were selected from shared hatching batches at the Anfu Waterfowl Breeding Base, Chongqing, China (latitude: 105.478° N, longitude: 29.343° E). The geese received a standardized diet, temperature, and lighting during feeding, with unrestricted access to water. When the geese reached 70 days of age, 215 healthy individuals were randomly selected as experimental subjects. Blood samples were collected from the wings of geese using vacuum tubes containing the anticoagulant EDTA and subsequently stored at −20 °C for future experimental use. Three fixed points of 215 right pectoral muscle samples of the goose slaughtered approximately 2 hours earlier were measured for three parameters of meat color (L*, a*, b*) using an automated colorimeter and a portable pH meter. Muscle samples were normalized for thickness and weight (M0) and then immersed in a thermostatic water bath at 80 °C (Model SW 22, Julabo GmbH, Seelbach, Germany). Once the central temperature of the samples reached 70 °C, the samples were incubated for 30 minutes. The samples were then removed from the heat source and cooled to room temperature. The samples were then weighed (M1) and the cooking loss rate (CLR) was calculated as CLR=(M0−M1)/M0*100%.

The cooled pectoral muscle was fixed perpendicular to the muscle fibers into long strips with a cross-sectional area of 1 cm×1 cm and sheared with a tenderness instrument (CLM-3) and repeated five times to obtain samples for mean shear force (SF). A 100 g sample of fresh meat was freeze-dried into powder, and the powder (Mp) was weighed, with the moisture content of freeze-dried (MCFD) calculated as MCFD=(100−Mp)/100*100%. Fat was extracted from 1 g of freeze-dried powder with petroleum benzene and the remaining material (M_{R}) was dried at 105 °C for 2 hours, with the crude fat content (CFC) then calculated as CFC=(1−M_{R}*Mp/100*100%).

### 1.2 Whole Genome Resequencing and SNP Calling

Genomic DNA was extracted from whole blood samples of the goose using the DP332 Genomic DNA Extraction Kit purchased from Tiangen Biotech. The quality of the extracted DNA was assessed by measuring the OD260/280 ratio ranging from 1.8 to 1.89. To facilitate Illumina sequencing, the original DNA samples were diluted to 50 ng/μL. The whole genome sequencing (WGS) was performed by Illumina HiSeq X Ten platform using 100 μl from each sample. Following generation of WGS data, quality control procedures were performed using FastQC and NGS QC toolkit software, which involved removing adapter sequences, residual primers, trimming low-quality bases. Filtered reads were aligned to the goose genome using BWA software (version ASM1303099v1) and SNPs were subsequently called using the GATK software. The trait phenotype dataset in the present study consisted of only individual-level samples with the completeness of genotype data exceeding 95%. The SNP filtering was performed using Plink, with the following parameters used at the SNP level: --genome 0.1-hwe 0.0000001-maf 0.05-mind 0.1. In addition, less than 10% of individuals with missing genotypes were retained. In total, 203 individuals were retained for further analysis. Following this, a principal component analysis (PCA) was performed to assess goose population stratification using the model implemented in Plink.

### 1.3 GWAS Analysis

The genome-wide association analysis (GWAS) using GEMMA software was performed to identify single nucleotide polymorphisms (SNPs) associated with various quality traits in goose meat, including CF, MCFD, CLR, SF, and meat color (L*, a*, b*). The GWAS analysis was performed using a mixed linear model expressed as the equation y=Wα+xβ+ε, where γ represents the phenotypic value of all individuals, W is the covariance matrix used to control the population structure, α is the coefficient vector including the intercept, variable x corresponds to the genotype of the SNP or haplotype marker, β represents the magnitude of the effect of the SNP or haplotype marker on the phenotype, and vector ε indicates the random residual. The importance of the association between SNPs and phenotypes was assessed by the Wald test and the Bonferroni correction was applied to adjust the results of association analysis. Manhattan plots, depicting association, were generated using GAP packages from https://cran.r-project.org/web/packages/gap/. In addition, Q-Q plots illustrating the distribution of p-values were created using the qqman package from https://cran.r-project.org/web/packages/qqman/in the R Project software, and in order to assess the presence of false-positive signals in the results obtained, the GenABEL package was utilized and the genome inflation factor (k) was calculated.

In order to identify SNP genotypes significantly associated with meat quality traits in Anser cygnoides domesticus by the GWAS analysis, amplification and extension primers were designed to amplify the target sequence and facilitate hybridization of fragments and extension of specific nucleotides of interest. To assess differences in meat quality traits among genotypes of selected SNPs, a general linear model was applied in the JMP software (version 13.0). Following this, least-squares means were calculated for each genotype and Bonferroni tests were performed to assess differences between genotypes.

In the present study, BEDTools software was used to extract genetic information from a 1 Mb region upstream and downstream of each potential SNP in the goose genome, along with Annovar software for SNP annotation (SnpEff, Annovar, VEP, Oncotator). Candidate genes were analyzed for functional annotation using the Metascape website (https://metascape.org/).

### 2 Results

### 2.1 Phenotypic Description of Goose Populations

The statistical data of meat quality traits in pectoral muscles of 70-day-old male Anser cygnoides domesticus in the present study are shown in the following table, including the crude fat content (CFC), meat color (L*, a* and b*), shear force (SF), moisture content of freeze-dried (MCFD), and cooking loss rate (CLR).

TABLE 1
Descriptive statistics on meat quality traits in 70-day-old
male Anser cygnoides domesticus

| | | | | | | CV |
|---|---|---|---|---|---|---|
| Traits | Number | Mean | STDV | Minimum | Maximum | (%) |
| CFC | 199 | 9.92 | 1.89 | 4.69 | 15.52 | 0.19 |
| MCFD | 199 | 75.07 | 0.94 | 72.26 | 77.54 | 0.01 |
| CLR | 194 | 13.13 | 3.01 | 4.66 | 22.36 | 0.23 |
| L*(meat | 205 | 23.6 | 3.65 | 15.53 | 37.29 | 0.15 |
| lightness) | | | | | | |
| a*(meat | 205 | 48.77 | 3.02 | 39.76 | 56.06 | 0.06 |
| redness) | | | | | | |
| b*(meat | 205 | 19.5 | 2.32 | 13.05 | 30.8 | 0.12 |
| yellowness) | | | | | | |
| SF (kgf) | 197 | 3.81 | 0.83 | 1.66 | 5.82 | 0.22 |

### 2.2 Whole Genome Resequencing and SNP Calling

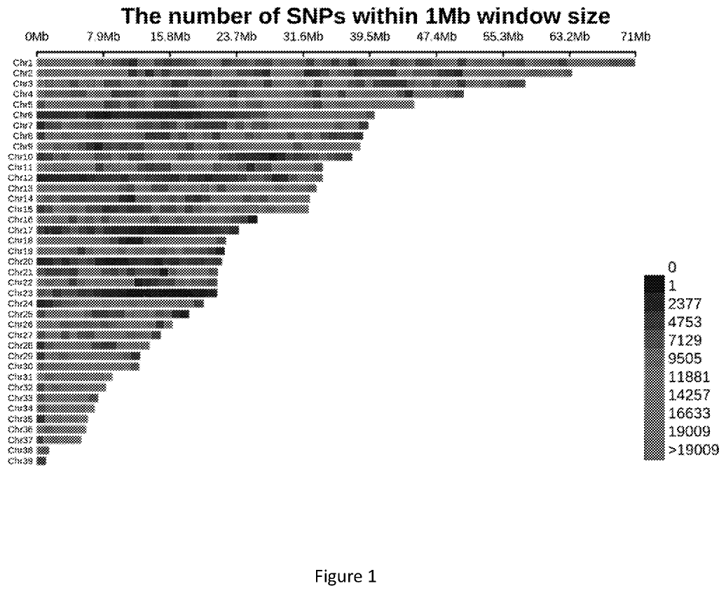

2,608.44 Gb of WGS data were obtained from 215 male Sichuan White Geese with an average depth coverage of 10.89 x. After screening, 2601.19 Gb of high-quality sequencing data were obtained, with the average mapping rate of 97.88% (97.72%~98.10%), the average Q20 and Q30 values of 96.77% and 91.90%, respectively, and the average GC content calculated to be 43.63% after alignment with the reference genome sequence of the goose. Subsequent analyses focused on the 10,072,006 SNPs identified in the goose genome (version ASM1303099v1) (FIG. 1).

### 2.3 Goose Population Structure and GWAS Analysis

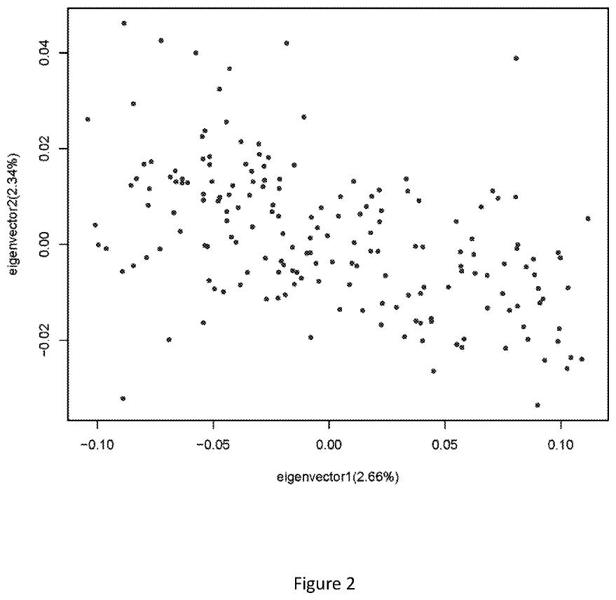

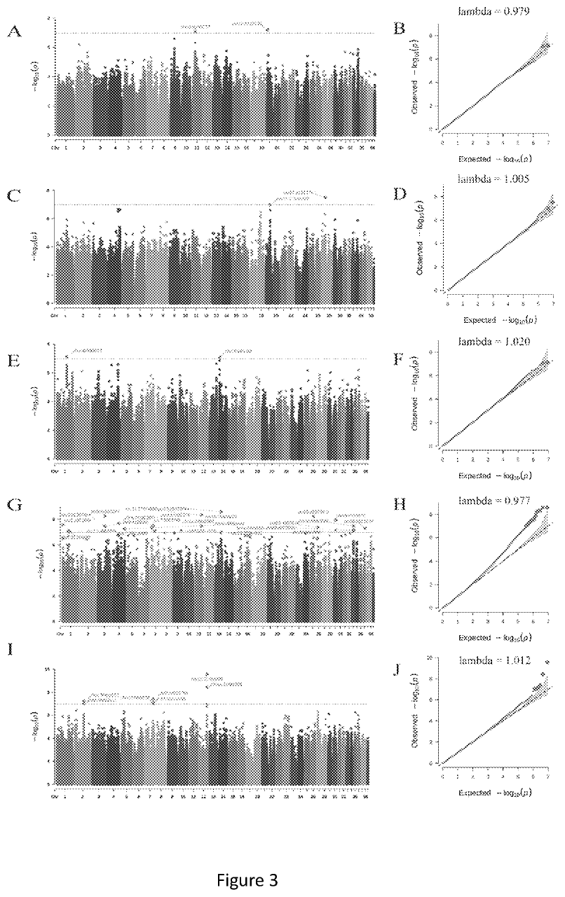

The PCA analysis indicated no significant population stratification (FIG. 2), so a mixed linear model was used to conduct a genome-wide association study (GWAS) of meat quality traits using the GEMMA software package. A total of 43 SNPs located in 28 genes (see Table below) were identified that showed significant associations with meat quality traits (FIG. 3).

TABLE 2
Summary of 43 SNP markers associated with five meat
quality traits in Anser cygnoides domesticus

| SNP | Chr | Position (bp) | allele1 | Traits | P value | Gene |
|---|---|---|---|---|---|---|
| chr11: 9193757 | chr11 | 9193757 | G/A | CFC | 8.12 × 10⁻⁰⁸ | TMEM65 |
| chr19: 2113530 | chr19 | 2113530 | C/T | CFC | 6.34 × 10⁻⁰⁸ | SMAD6 |
| chr19: 17076992 | chr19 | 17076992 | T/G | CLR | 1.03 × 10⁻⁰⁷ | SYTC2 |
| chr27: 11913710 | chr27 | 11913710 | G/A | CLR | 3.16 × 10⁻⁰⁸ | MSI1H |
| chr1: 42733613 | chr1 | 42733613 | T/C | L*(meat lightness) | 7.56 × 10⁻⁰⁸ | LIN1 |
| chr14: 440052 | chr14 | 440052 | T/C | L*(meat lightness) | 8.15 × 10⁻⁰⁸ | GSS |
| chr1: 1971942 | chr1 | 1971942 | C/T | b*(meat yellowness) | 2.74 × 10⁻⁰⁸ | EXTL3 |
| chr1: 24867798 | chr1 | 24867798 | C/G | b*(meat yellowness) | 7.82 × 10⁻⁰⁸ | PRSS55 |
| chr1: 31137893 | chr1 | 31137893 | C/T | b*(meat yellowness) | 9.31 × 10⁻⁰⁸ | PTP4A1 |
| chr3: 31387295 | chr3 | 31387295 | G/A | b*(meat yellowness) | 5.43 × 10⁻⁰⁹ | TMEM19 |
| chr3: 31387314 | chr3 | 31387314 | G/A | b*(meat yellowness) | 3.57 × 10⁻⁰⁸ | TMEM19 |
| chr3: 31387324 | chr3 | 31387324 | A/G | b*(meat yellowness) | 3.57 × 10⁻⁰⁸ | TMEM19 |
| chr7: 8983345 | chr7 | 8983345 | A/G | b*(meat yellowness) | 3.20 × 10⁻⁰⁸ | ACKR3 |
| chr7: 16992800 | chr7 | 16992800 | C/T | b*(meat yellowness) | 7.84 × 10⁻⁰⁸ | CHIN |
| chr7: 4199190 | chr7 | 4199190 | A/G | b*(meat yellowness) | 4.47 × 10⁻⁰⁸ | GULP1 |
| chr9: 33911228 | chr9 | 33911228 | A/G | b*(meat yellowness) | 7.35 × 10⁻⁰⁸ | DOCK1 |
| chr11: 27962909 | chr11 | 27962909 | A/G | b*(meat yellowness) | 3.43 × 10⁻⁰⁸ | PP4R1 |
| chr11: 27964588 | chr11 | 27964588 | G/T | b*(meat yellowness) | 4.66 × 10⁻⁰⁹ | SLCO5A1 |
| chr13: 25432644 | chr13 | 25432644 | C/T | b*(meat yellowness) | 2.55 × 10⁻⁰⁹ | CATC |
| chr13: 25426268 | chr13 | 25426268 | G/A | b*(meat yellowness) | 2.62 × 10⁻⁰⁹ | CATC |
| chr13: 25389761 | chr13 | 25389761 | T/G | b*(meat yellowness) | 9.87 × 10⁻⁰⁸ | CATC |
| chr26: 4307814 | chr26 | 4307814 | T/C | b*(meat yellowness) | 4.26 × 10⁻⁰⁸ | PP4R1 |
| chr29: 8720675 | chr29 | 8720675 | T/C | b*(meat yellowness) | 1.16 × 10⁻⁰⁸ | MRPS2 |
| chr33: 5370910 | chr33 | 5370910 | A/C | b*(meat yellowness) | 6.90 × 10⁻⁰⁸ | NO40 |
| chr33: 5659799 | chr33 | 5659799 | A/C | b*(meat yellowness) | 3.33 × 10⁻⁰⁸ | PUM1 |
| chr33: 3667950 | chr33 | 3667950 | C/T | b*(meat yellowness) | 8.99 × 10⁻⁰⁸ | RRAGC |
| chr36: 5611038 | chr36 | 5611038 | G/T | b*(meat yellowness) | 4.99 × 10⁻⁰⁸ | GOGA7 |
| ctg2092: 176333 | ctg2092 | 176333 | C/T | b*(meat yellowness) | 6.16 × 10⁻⁰⁸ | RXRA |
| ctg745: 127150 | ctg745 | 127150 | A/G | b*(meat yellowness) | 5.53 × 10⁻⁰⁸ | NCOA2 |
| chr4: 23787309 | chr4 | 23787309 | A/G | b*(meat yellowness) | 2.08 × 10⁻⁰⁸ | None |
| chr4: 49863664 | chr4 | 49863664 | T/A | b*(meat yellowness) | 5.49 × 10⁻⁰⁸ | None |
| chr25: 12875901 | chr25 | 12875901 | A/G | b*(meat yellowness) | 5.77 × 10⁻⁰⁹ | None |
| chr29: 8278595 | chr29 | 8278595 | C/T | b*(meat yellowness) | 6.64 × 10⁻⁰⁸ | None |
| ctg834: 24329 | ctg834 | 24329 | C/T | b*(meat yellowness) | 5.15 × 10⁻⁰⁸ | None |
| ctg930: 11921 | ctg930 | 11921 | G/A | b*(meat yellowness) | 5.30 × 10⁻⁰⁹ | None |
| ctg956: 92804 | ctg956 | 92804 | C/T | b*(meat yellowness) | 2.73 × 10⁻⁰⁸ | None |
| chr2: 36812553 | chr2 | 36812553 | C/T | SF (kgf) | 5.94 × 10⁻⁰⁸ | BMF |
| chr2: 36812412 | chr2 | 36812412 | G/C | SF (kgf) | 9.22 × 10⁻⁰⁸ | BMF |
| chr7: 33558795 | chr7 | 33558795 | A/T | SF (kgf) | 3.84 × 10⁻⁰⁸ | NEMP2 |
| chr7: 33558758 | chr7 | 33558758 | C/A | SF (kgf) | 8.04 × 10⁻⁰⁸ | NEMP2 |
| chr7: 33558832 | chr7 | 33558832 | T/C | SF (kgf) | 9.30 × 10⁻⁰⁸ | NEMP2 |
| chr12: 31782870 | chr12 | 31782870 | A/G | SF (kgf) | 2.78 × 10⁻¹⁰ | NAD-ME |
| chr12: 31781825 | chr12 | 31781825 | G/A | SF (kgf) | 3.62 × 10⁻⁰⁹ | NAD-ME |

Note:
The significance threshold is set at 1 × 10⁻⁷.
ACKR3: atypical

### DESCRIPTION

chemokine receptor 3; BMF: Bcl-2-modifier; CATC: dipeptidyl peptidase 1; CHIN: N-chimeric protein; DOCK1: dedicator of cytokinesis 1; EXTL3: exosin-like 3; GOGA7: Golgin subfamily A member 7; GSS: glutamine synthetase; GULP1: GULP PTB domain containing engulfment adaptor 1; LIN1: LINE-1 reverse transcriptase homolog; MRPS2: 28S ribosomal protein S2, mitochondrial; MSI1H: RNA-binding protein Musashi homolog 1; NAD-ME: NAD-dependent malic enzyme, mitochondrial; NCOA2: nuclear receptor coactivator 2; NEMP2: nuclear membrane integral membrane protein 2; NO40: nucleolar protein of 40 kDa; PP4R1: serine/threonine-protein phosphatase 4 regulatory subunit 1; PRSS55: serine protease 55; PTP4A1: protein tyrosine phosphatase type IVA 1; PUM1: pumilio homologue 1; RRAGC: Ras-related GTP-binding protein C; RXRA: retinoic acid receptor RXR-a; SLCO5A1: solute carrier organic anion transporter family member 5A1; SMAD6: mothers against decapentaplegic homolog 6; SYTC2: possible threonine-tRNA ligase 2, cytoplasmic; TMEM19: transmembrane protein 19; TMEM65: transmembrane protein 65; None: No annotated gene was found in a specific region of the genome.

### 2.4 Genotypes of Selected SNPs

Eleven SNPs genotypes were examined and meat quality traits were compared across genotypes identified by GWAS (Table 3-4), including eight SNPs (chr13:25432644, ctg930:11921, chr36:5611038, ctg2092:176333, chr29:8278595, chr9:33911228, chr1:24867798, and chr7:16992800) that were significantly associated with b* meat yellowness, and three SNPs (chr12:31782870, chr12:31781825, and chr2:36812553) that were significantly associated with SF meat quality traits.

TABLE 3
Primer sequences of MALDI-TOF MS

| SNPs | 2nd-PCRP | 2nd-PCRP | UEP_SEQ |
|---|---|---|---|
| chr13: | ACGTTGGATGGAAAT | ACGTTGGATGACCTG | agaaCCTGTTGTCTC |
| 25432644 | ACCCTGTTGTCTCCC | TTTGACTCCTTTTGG | CCTCACTC |
| ctg930: | ACGTTGGATGTGCCA | ACGTTGGATGTGGCA | GCCCCCTCCTGCACC |
| 11921 | CCGCAGGGATCACG | GCAGGGTGGGGAAA | TT |
| chr36: | ACGTTGGATGTTCCC | ACGTTGGATGCCCAG | tCGCTTTGATTTAGT |
| 5611038 | CCCTCGTTTGAATTG | TCTGAATTCCAACAC | TATTTTACTC |
| ctg2092: | ACGTTGGATGGGAAT | ACGTTGGATGTTTTG | cccaTAGACTACGTC |
| 176333 | ATGTAGACTACGTCT | GACAAACAGGAGACC | TGCCATCT |
| | G | | |
| chr29: | ACGTTGGATGAAGGA | ACGTTGGATGTGCAC | CAGGAACCGAGGGAA |
| 8278595 | TTTGGGAAGCAGGAA | CGGGGAGGAGGAGA | ATGC |
| | C | | |
| chr9: | ACGTTGGATGCCTGG | ACGTTGGATGCTTAA | CAGGAGTTAAGGGAG |
| 33911228 | AGGCAATCAAAGATG | GTCGCCTTGGTACAC | AAAAT |
| chr1: | ACGTTGGATGAGCCT | ACGTTGGATGGTGTG | GCTGCCTGCCCAGAA |
| 24867798 | GATGCAGTCACATCC | TGCCAGACAAAACTC | CT |
| chr7: | ACGTTGGATGTCACA | ACGTTGGATGTTATA | gtCAGGGTCCAACTC |
| 16992800 | TTGGCAGGGTCCAAC | GTCTGCTCTGGACTG | AGTCTCC |
| chr12: | ACGTTGGATGACAAA | ACGTTGGATGGGCAG | CAATAATGTTTAACG |
| 31782870 | GAACACATCGCAAGG | CAGCTTTCAGCAAAC | TTAGACTC |
| chr12: | ACGTTGGATGTTCTG | ACGTTGGATGTTTAA | CCAAAGACCTGTTGA |
| 31781825 | CAACGCTCGAAATCC | CCTACGCATGCCTCC | GA |
| chr2: | ACGTTGGATGGTGGA | ACGTTGGATGGCAGG | tcGATCTTACTTTTT |
| 36812553 | AGAAGACATCACTGG | AGAAAAAAGCATAAG | TATCTTCCATTA |

TABLE 4
Frequencies and genotypes of selected SNPs associated with goose meat quality

| Traits | Number | SNPs | Genotypes (Frequency %) | | |
|---|---|---|---|---|---|
| b*(meat yellowness) | 215 | chr13:25432644 | CC | CT | TT |
| | | | 17.61 ± 2.07^{b} (1.97) | 17.56 ± 2.92^{b} (0.66) | 19.31 ± 0.25^{a} (97.37) |
| b*(meat yellowness) | 215 | ctg930:11921 | AA | GA | GG |
| | | | 19.18 ± 0.23^{b} (72.37) | 20.28 ± 0.41^{a} (23.03) | 19.03 ± 0.88^{b} (4.61) |
| b*(meat yellowness) | 215 | chr36:5611038 | GG | GT | TT |
| | | | 19.83 ± 2.76^{a} (0.58) | 17.71 ± 1.04^{b} (4.62) | 19.44 ± 0.22^{a} (94.80) |
| b*(meat yellowness) | 215 | ctg2092:176333 | CC | TC | TT |
| | | | 18.04 ± 1.15^{b} (3.49) | 19.24 ± 0.45^{a} (13.56) | 19.48 ± 0.26^{a} (70.93) |
| b*(meat yellowness) | 215 | chr29:8278595 | CC | CT | TT |
| | | | 17.99 ± 2.80^{b} (0.56) | 19.21 ± 0.60^{a} (13.56) | 19.39 ± 0.23^{a} (85.88) |
| b*(meat yellowness) | 215 | chr9:33911228 | AA | GA | GG |
| | | | 20.32 ± 1.62^{a} (2.27) | 19.52 ± 0.75^{b} (8.52) | 19.31 ± 0.23^{b} (89.20) |
| b*(meat yellowness) | 215 | chr1:24867798 | CC | GC | GG |
| | | | 16.33 ± 2.79^{b} (0.57) | 19.08 ± 0.72^{a} (9.09) | 19.41 ± 0.23^{a} (90.34) |
| b*(meat yellowness) | 215 | chr7:16992800 | CC | CT | TT |
| | | | 15.43 ± 2.77^{b} (1.12) | 19.30 ± 0.57^{a} (13.48) | 19.40 ± 0.23^{a} (85.39) |
| SF | 215 | chr12:31782870 | AA | GA | GG |
| | | | 3.12 ± 0.97^{b} (1.12) | 3.82 ± 0.25^{a} (8.43) | 3.74 ± 0.08^{a} (90.45) |
| SF | 215 | chr12:31781825 | AA | GA | GG |
| | | | 3.72 ± 0.08^{b} (87.01) | 3.89 ± 0.22^{a} (12.99) | \ |
| SF | 215 | chr2:36812553 | CC | CT | TT |
| | | | \ | 3.60 ± 0.22^{b} (15.49) | 3.73 ± 0.09^{a} (84.51) |

Note:
Different letters in the same column indicate significant differences between haplotypes (P < 0.05).

2.5 Gene Annotation within 1 Mb of Significant SNPs

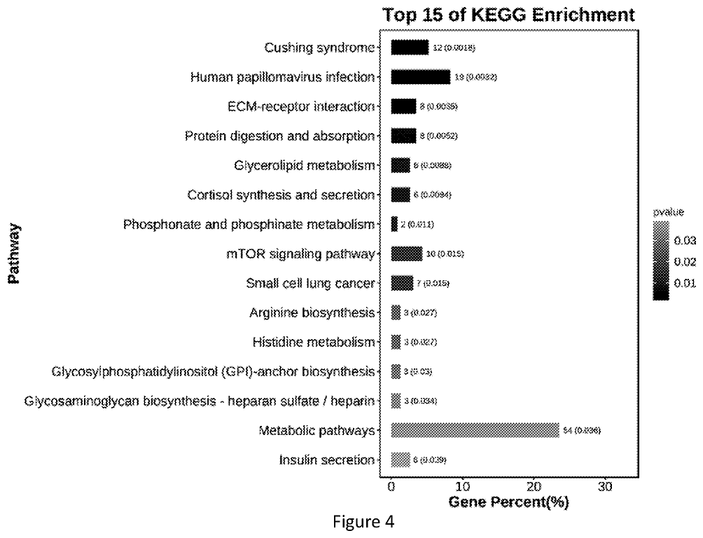

Significant SNPs with quality traits in goose meat were annotated within 1 Mb using the KEGG database. The gene enrichment analysis showed that genes were enriched in Cushing syndrome (ko04934, p-value=0.001797367), human papillomavirus infection (ko05165, p-value=0.003221264), ECM-receptor interaction (ko04512, p-value=0.003526523), protein digestion and absorption (ko04974, p-value=0.00521013), and glycerolipid metabolism (ko00561, p-value=0.008786627) (FIG. 4).

### 3 Discussion

The Anser cygnoides domesticus, known for meat and egg production, is a dual-purpose breed of poultry in China and is a valuable genetic resource that has made significant contributions to the poultry industry, bringing enormous economic and nutritional benefits to China. Meat quality in the poultry industry is a key factor in consumer acceptance and ultimately the economic success of the industry. Poultry breeders use selective breeding and genetic improvement programs to enhance meat quality traits with the aim of producing poultry that meets consumer preferences and needs. In the present study, meat quality traits of 70-day-old male Anser cygnoides domesticus were investigated. Using GWAS, a total of 43 SNPs associated with CFC, CLR, meat color, and SF traits were screened and identified in 215 Chinese indigenous geese (Anser cygnoides domesticus), with significant associations between these SNPs and the above traits determined by the GWAS analysis.

Sample size is a key factor in research design that affects the validity and reliability of the results. For larger sample sizes, results can be improved by reducing sampling error, increasing statistical power, and improving generalizability and precision. In the present study, the principal component analysis indicated no significant stratification in the population (FIG. 2), and Q-Q plots indicated a good fit between observed and predicted values, thus showing no significant population stratification. In addition, 11 SNP loci associated with SNPs of meat quality were identified using MALDI-TOP MS and the frequencies of candidate SNPs were analyzed, which corresponded to the results of the GWAS analysis. In summary, the results of the present study are valid and reliable, and SNPs can be used as breeding markers in meat geese.

The crude fat content (CFC) of meat is a crucial indicator of meat quality and has been shown to correlate with several other quality characteristics. In the present study, genetic factors for CFC in geese were investigated and two important SNPs located in the transmembrane protein 65 (TMEM65) and Mothers against decapentaplegic homolog 6 (SMAD6) were identified. TMEM65 is a mitochondrial inner membrane protein that plays a crucial role in the oxidative stress response, oxygen consumption, and citrate synthase activity. Since CFC is closely associated with fat anabolism affected by the oxidative function of mitochondria, it is hypothesized that TMEM65 may be involved in fat oxidative metabolism and affect CFC levels indirectly. The metabolism of fatty acids has a significant effect on flavor and intramuscular fat production, which in turn affects meat tenderness, water retention, and flavor presentation. Therefore, the identification of genetic markers associated with CFC, such as SNPs identified in TMEM65 in the present study, may be important for the selection and breeding of animals with desirable meat quality traits.

Meat color (L*, a*, and b*), as an important indicator for evaluating meat appearance, reflects changes in muscle physiology, biochemistry, and microbiology, affecting the functional properties and quality of highly processed meat products. In the present study (Table 2), 30 SNPs significantly associated with meat color (MC) were identified, among which glutamine synthetase (GSS) is one of the identified candidate genes that plays a crucial role in several cellular processes with the main function of producing glutathione, which protects the cells from oxidative damage and aids in amino acid translocation. In addition, GSS are involved in the detoxification of foreign compounds. The presence of GSS as a candidate gene suggests its importance in cell protection and maintenance of cellular homeostasis, and further exploration of the role of GSS in these processes will provide valuable insights into their contribution to cellular health and their potential impact on various biological functions. Ras-related GTP-binding protein C (RRAGC) and solute carrier organic anion transporter family member 5A1 (SLCO5A1) play crucial roles in cellular responses to the availability of amino acids. RRAGC, along with three other Rag proteins, also plays a critical role in the activation of the mTORC1 pathway, which is sensitive to amino acid levels, regulating metabolic and physiological processes such as lipid metabolism, autophagy, and muscle protein synthesis. Following slaughter, oxygen supplies to cells decrease, resulting in accelerated adenosine triphosphate (ATP) breakdown, increased free radicals, lactate accumulation, and changes in pH. These redox reactions promote protein denaturation and increase fluid outflow, resulting in changes in meat color and water retention capacity, which in turn cause changes in meat color and water retention. Therefore, it is hypothesized that GSS, RRAGC, and SLCO5A1 may be responsible for carcass color presentation through oxidative metabolism.

The shear force (SF) is a key parameter in assessing meat tenderness and is influenced by several factors, such as muscle fiber properties (e.g., diameter, density, type, and integrity), components of connective tissue (e.g., content, type, and cross-linking status), properties of myogenic fibrinolytic enzyme, and intramuscular fat content. In the present study, seven SNPs located in Bcl-2 modifier (BMF), nuclear membrane integral membrane protein 2 (NEMP2), and NAD-dependent malic enzyme, mitochondrial (NAD-ME) were identified, which were significantly associated with SF in geese. The association was further verified by the results of MALDI-TOF MS. In the present study, a significant association was confirmed between the genotype (chr2:36812553) of the SNP in BMF as well as two SNPs (chr12:31782870, chr12:31781825) in NAD-ME and the shear force (SF) in goose meat. BMF, a member of the Bcl2 protein family, is an intracellular damage sensor that triggers apoptosis and may mediate muscle fiber atrophy. NAD-ME is involved in various metabolic pathways that provide the reducing power (NADPH) for the synthesis of various cellular components, and in poultry, NAD-ME serves as a source of energy for the synthesis of long-chain fatty acids in the poultry liver. It is hypothesized that BMF may directly affect myofiber structure, whereas NAD-ME may affect oxidoreductase activity in muscle and intramuscular lipogenesis, ultimately affecting SF.

The SNPs in the GWAS analysis were not previously reported for the following three reasons: First, in the present study, the goose genome sequence (version ASM1303099v1) at the first chromosome level was utilized as a reference, which improved genome assembly and annotation compared to previous studies. In addition, the lack of similar studies on data of goose meat quality, as well as the small changes experienced by geese during domestication from wild migratory birds, led to the lack of reports on many other genes associated with meat quality in the present study.

### 4 Conclusion

In the present study, the GWAS analysis was performed for quality traits in goose meat, with 43 SNPs significantly associated with these traits identified. In addition, the selected SNPs associated with traits in the same population were genotyped using the MALDI-TOP MS method. The results of the present study revealed 28 annotated genes that showed promise as potential candidate genes associated with the five meat quality traits studied, and which also represent new genetic markers that could be implemented in marker-assisted selection programs for geese. Importantly, the present study contributes to a comprehensive understanding of the genetic mechanisms underlying quality traits in goose meat and lays a solid foundation for future research in this field. The various technical features of the above embodiments can be combined in any way, and not all possible combinations of the technical features in the above embodiments are described one by one for the sake of conciseness of description. However, as long as there is no contradiction in the combinations of these technical features, all the combinations are intended to be embraced.

The specific and detailed embodiments described above are merely exemplary in nature and is in no way intended to limit the PRESENT invention. It will be apparent to those skilled in the art that modifications and variations could be made without departing from the scope of the present invention as defined by the appended claims.

What is claimed is:

1. A SNP molecular marker associated with quality traits in goose meat, wherein the SNP molecular marker is as follows:

| SNP | Chr | Position (bp) | allele1 | Traits | P value | Gene |
|---|---|---|---|---|---|---|
| chr11: 9193757 | chr11 | 9193757 | G/A | CFC | 8.12 × 10⁻⁰⁸ | TMEM65 |
| chr19: 2113530 | chr19 | 2113530 | C/T | CFC | 6.34 × 10⁻⁰⁸ | SMAD6 |
| chr19: 17076992 | chr19 | 17076992 | T/G | CLR | 1.03 × 10⁻⁰⁷ | SYTC2 |
| chr27: 11913710 | chr27 | 11913710 | G/A | CLR | 3.16 × 10⁻⁰⁸ | MSI1H |
| chr1: 42733613 | chr1 | 42733613 | T/C | L*(meat lightness) | 7.56 × 10⁻⁰⁸ | LIN1 |
| chr14: 440052 | chr14 | 440052 | T/C | L*(meat lightness) | 8.15 × 10⁻⁰⁸ | GSS |
| chr1: 1971942 | chr1 | 1971942 | C/T | b*(meat yellowness) | 2.74 × 10⁻⁰⁸ | EXTL3 |
| chr1: 24867798 | chr1 | 24867798 | C/G | b*(meat yellowness) | 7.82 × 10⁻⁰⁸ | PRSS55 |
| chr1: 31137893 | chr1 | 31137893 | C/T | b*(meat yellowness) | 9.31 × 10⁻⁰⁸ | PTP4A1 |
| chr3: 31387295 | chr3 | 31387295 | G/A | b*(meat yellowness) | 5.43 × 10⁻⁰⁹ | TMEM19 |
| chr3: 31387314 | chr3 | 31387314 | G/A | b*(meat yellowness) | 3.57 × 10⁻⁰⁸ | TMEM19 |
| chr3: 31387324 | chr3 | 31387324 | A/G | b*(meat yellowness) | 3.57 × 10⁻⁰⁸ | TMEM19 |
| chr7: 8983345 | chr7 | 8983345 | A/G | b*(meat yellowness) | 3.20 × 10⁻⁰⁸ | ACKR3 |
| chr7: 16992800 | chr7 | 16992800 | C/T | b*(meat yellowness) | 7.84 × 10⁻⁰⁸ | CHIN |
| chr7: 4199190 | chr7 | 4199190 | A/G | b*(meat yellowness) | 4.47 × 10⁻⁰⁸ | GULP1 |
| chr9: 33911228 | chr9 | 33911228 | A/G | b*(meat yellowness) | 7.35 × 10⁻⁰⁸ | DOCK1 |
| chr11: 27962909 | chr11 | 27962909 | A/G | b*(meat yellowness) | 3.43 × 10⁻⁰⁸ | PP4R1 |
| chr11: 27964588 | chr11 | 27964588 | G/T | b*(meat yellowness) | 4.66 × 10⁻⁰⁹ | SLCO5A1 |
| chr13: 25432644 | chr13 | 25432644 | C/T | b*(meat yellowness) | 2.55 × 10⁻⁰⁹ | CATC |
| chr13: 25426268 | chr13 | 25426268 | G/A | b*(meat yellowness) | 2.62 × 10⁻⁰⁹ | CATC |
| chr13: 25389761 | chr13 | 25389761 | T/G | b*(meat yellowness) | 9.87 × 10⁻⁰⁸ | CATC |
| chr26: 4307814 | chr26 | 4307814 | T/C | b*(meat yellowness) | 4.26 × 10⁻⁰⁸ | PP4R1 |
| chr29: 8720675 | chr29 | 8720675 | T/C | b*(meat yellowness) | 1.16 × 10⁻⁰⁸ | MRPS2 |
| chr33: 5370910 | chr33 | 5370910 | A/C | b*(meat yellowness) | 6.90 × 10⁻⁰⁸ | NO40 |
| chr33: 5659799 | chr33 | 5659799 | A/C | b*(meat yellowness) | 3.33 × 10⁻⁰⁸ | PUM1 |
| chr33: 3667950 | chr33 | 3667950 | C/T | b*(meat yellowness) | 8.99 × 10⁻⁰⁸ | RRAGC |
| chr36: 5611038 | chr36 | 5611038 | G/T | b*(meat yellowness) | 4.99 × 10⁻⁰⁸ | GOGA7 |
| ctg2092: 176333 | ctg2092 | 176333 | C/T | b*(meat yellowness) | 6.16 × 10⁻⁰⁸ | RXRA |
| ctg745: 127150 | ctg745 | 127150 | A/G | b*(meat yellowness) | 5.53 × 10⁻⁰⁸ | NCOA2 |
| chr4: 23787309 | chr4 | 23787309 | A/G | b*(meat yellowness) | 2.08 × 10⁻⁰⁸ | None |
| chr4: 49863664 | chr4 | 49863664 | T/A | b*(meat yellowness) | 5.49 × 10⁻⁰⁸ | None |
| chr25: 12875901 | chr25 | 12875901 | A/G | b*(meat yellowness) | 5.77 × 10⁻⁰⁹ | None |
| chr29: 8278595 | chr29 | 8278595 | C/T | b*(meat yellowness) | 6.64 × 10⁻⁰⁸ | None |
| ctg834: 24329 | ctg834 | 24329 | C/T | b*(meat yellowness) | 5.15 × 10⁻⁰⁸ | None |
| ctg930: 11921 | ctg930 | 11921 | G/A | b*(meat yellowness) | 5.30 × 10⁻⁰⁹ | None |
| ctg956: 92804 | ctg956 | 92804 | C/T | b*(meat yellowness) | 2.73 × 10⁻⁰⁸ | None |
| chr2: 36812553 | chr2 | 36812553 | C/T | SF (kgf) | 5.94 × 10⁻⁰⁸ | BMF |
| chr2: 36812412 | chr2 | 36812412 | G/C | SF (kgf) | 9.22 × 10⁻⁰⁸ | BMF |
| chr7: 33558795 | chr7 | 33558795 | A/T | SF (kgf) | 3.84 × 10⁻⁰⁸ | NEMP2 |
| chr7: 33558758 | chr7 | 33558758 | C/A | SF (kgf) | 8.04 × 10⁻⁰⁸ | NEMP2 |
| chr7: 33558832 | chr7 | 33558832 | T/C | SF (kgf) | 9.30 × 10⁻⁰⁸ | NEMP2 |
| chr12: 31782870 | chr12 | 31782870 | A/G | SF (kgf) | 2.78 × 10⁻¹⁰ | NAD-ME |
| chr12: 31781825 | chr12 | 31781825 | G/A | SF (kgf) | 3.62 × 10⁻⁰⁹ | NAD-ME |

2. The SNP molecular marker associated with quality traits in goose meat according to claim 1, wherein 11 SNP genotypes comprise:
genotypes associated with b* meat yellowness:
chr13:25432644;
chr13:25432644;
ctg930:11921;
chr36:5611038;
ctg2092:176333;
chr29:8278595;
chr9:33911228;
chr1:24867798;
chr7:16992800;
genotypes associated with SF meat:
chr12:31782870;
chr12:31781825;
chr2:36812553.

3. The SNP molecular marker associated with quality traits in goose meat according to claim 1, wherein primer sequences of MALDI-TOF MS are as follows:

| SNPs | 2nd-PCRP | 2nd-PCRP | UEP_SEQ |
|---|---|---|---|
| chr13: | ACGTTGGATGGAAAT | ACGTTGGATGACCTG | agaaCCTGTTGTCTC |
| 25432644 | ACCCTGTTGTCTCCC | TTTGACTCCTTTTGG | CCTCACTC |
| ctg930: | ACGTTGGATGTGCCA | ACGTTGGATGTGGCA | GCCCCCTCCTGCACC |
| 11921 | CCGCAGGGATCACG | GCAGGGTGGGGAAA | TT |
| chr36: | ACGTTGGATGTTCCC | ACGTTGGATGCCCAG | tCGCTTTGATTTAGT |
| 5611038 | CCCTCGTTTGAATTG | TCTGAATTCCAACAC | TATTTTACTC |
| ctg2092: | ACGTTGGATGGGAAT | ACGTTGGATGTTTTG | cccaTAGACTACGTC |
| 176333 | ATGTAGACTACGTCT | GACAAACAGGAGACC | TGCCATCT |
| | G | | |
| chr29: | ACGTTGGATGAAGGA | AACACGTTGGATGTG | CAGGAACCGAGGGAA |
| 8278595 | TTTGGGAAGCAGG | CACCGGGGAGGAGGA | ATGC |
| | | GA | |
| chr9: | ACGTTGGATGCCTGG | ACGTTGGATGCTTAA | CAGGAGTTAAGGGAG |
| 33911228 | AGGCAATCAAAGATG | GTCGCCTTGGTACAC | AAAAT |
| chr1: | ACGTTGGATGAGCCT | ACGTTGGATGGTGTG | GCTGCCTGCCCAGAA |
| 24867798 | GATGCAGTCACATCC | TGCCAGACAAAACTC | CT |
| chr7: | ACGTTGGATGTCACA | ACGTTGGATGTTATA | gtCAGGGTCCAACTC |
| 16992800 | TTGGCAGGGTCCAAC | GTCTGCTCTGGACTG | AGTCTCC |
| chr12: | ACGTTGGATGACAAA | ACGTTGGATGGGCAG | CAATAATGTTTAACG |
| 31782870 | GAACACATCGCAAGG | CAGCTTTCAGCAAAC | TTAGACTC |
| chr12: | ACGTTGGATGTTCTG | ACGTTGGATGTTTAA | CCAAAGACCTGTTGA |
| 31781825 | CAACGCTCGAAATCC | CCTACGCATGCCTCC | GA |
| chr2: | ACGTTGGATGGTGGA | ACGTTGGATGGCAGG | tcGATCTTACTTTTT |
| 36812553 | AGAAGACATCACTGG | AGAAAAAAGCATAAG | TATCTTCCATTA |

4. The SNP molecular marker associated with quality traits in goose meat according to claim 1, wherein frequencies and genotypes of selected SNPs associated with goose meat quality are as follows:

| Traits | Number | SNPs | Genotypes (Frequency %) | | |
|---|---|---|---|---|---|
| b*(meat yellowness) | 215 | chr13:25432644 | CC | CT | TT |
| | | | 17.61 ± 2.07^{b} (1.97) | 17.56 ± 2.92^{b} (0.66) | 19.31 ± 0.25^{a} (97.37) |
| b*(meat yellowness) | 215 | ctg930:11921 | AA | GA | GG |
| | | | 19.18 ± 0.23^{b} (72.37) | 20.28 ± 0.41^{a} (23.03) | 19.03 ± 0.88^{b} (4.61) |
| b*(meat yellowness) | 215 | chr36:5611038 | GG | GT | TT |
| | | | 19.83 ± 2.76^{a} (0.58) | 17.71 ± 1.04^{b} (4.62) | 19.44 ± 0.22^{a} (94.80) |
| b*(meat yellowness) | 215 | ctg2092:176333 | CC | TC | TT |
| | | | 18.04 ± 1.15^{b} (3.49) | 19.24 ± 0.45^{a} (13.56) | 19.48 ± 0.26^{a} (70.93) |
| b*(meat yellowness) | 215 | chr29:8278595 | CC | CT | TT |
| | | | 17.99 ± 2.80^{b} (0.56) | 19.21 ± 0.60^{a} (13.56) | 19.39 ± 0.23^{a} (85.88) |
| b*(meat yellowness) | 215 | chr9:33911228 | AA | GA | GG |
| | | | 20.32 ± 1.62^{a} (2.27) | 19.52 ± 0.75^{b} (8.52) | 19.31 ± 0.23^{b} (89.20) |
| b*(meat yellowness) | 215 | chr1:24867798 | CC | GC | GG |
| | | | 16.33 ± 2.79^{b} (0.57) | 19.08 ± 0.72^{a} (9.09) | 19.41 ± 0.23^{a} (90.34) |
| b*(meat yellowness) | 215 | chr7:16992800 | CC | CT | TT |
| | | | 15.43 ± 2.77^{b} (1.12) | 19.30 ± 0.57^{a} (13.48) | 19.40 ± 0.23^{a} (85.39) |
| SF | 215 | chr12:31782870 | AA | GA | GG |
| | | | 3.12 ± 0.97^{b} (1.12) | 3.82 ± 0.25^{a} (8.43) | 3.74 ± 0.08^{a} (90.45) |
| SF | 215 | chr12:31781825 | AA | GA | GG |
| | | | 3.72 ± 0.08^{b} (87.01) | 3.89 ± 0.22^{a} (12.99) | \ |
| SF | 215 | chr2:36812553 | CC | CT | TT |
| | | | \ | 3.60 ± 0.22^{b} (15.49) | 3.73 ± 0.09^{a} (84.51) |


SEQUENCE LISTING (33 sequences)

SEQ ID NO: 1 - DNA, 30 bp, synthetic construct
  source 1..30: mol_type = other DNA
acgttggatggaaataccctgttgtctccc

SEQ ID NO: 2 - DNA, 30 bp, synthetic construct
  source 1..30: mol_type = other DNA
acgttggatgacctgtttgactccttttgg

SEQ ID NO: 3 - DNA, 23 bp, synthetic construct
  source 1..23: mol_type = other DNA
agaacctgttgtctccctcactc

SEQ ID NO: 4 - DNA, 29 bp, synthetic construct
  source 1..29: mol_type = other DNA
acgttggatgtgccaccgcagggatcacg

SEQ ID NO: 5 - DNA, 29 bp, synthetic construct
  source 1..29: mol_type = other DNA
acgttggatgtggcagcagggtggggaaa

SEQ ID NO: 6 - DNA, 17 bp, synthetic construct
  source 1..17: mol_type = other DNA
gccccctcctgcacctt

SEQ ID NO: 7 - DNA, 30 bp, synthetic construct
  source 1..30: mol_type = other DNA
acgttggatgttcccccctcgtttgaattg

SEQ ID NO: 8 - DNA, 30 bp, synthetic construct
  source 1..30: mol_type = other DNA
acgttggatgcccagtctgaattccaacac

SEQ ID NO: 9 - DNA, 25 bp, synthetic construct
  source 1..25: mol_type = other DNA
tcgctttgatttagttattttactc

SEQ ID NO: 10 - DNA, 31 bp, synthetic construct
  source 1..31: mol_type = other DNA
acgttggatgggaatatgtagactacgtctg

SEQ ID NO: 11 - DNA, 30 bp, synthetic construct
  source 1..30: mol_type = other DNA
acgttggatgttttggacaaacaggagacc

SEQ ID NO: 12 - DNA, 23 bp, synthetic construct
  source 1..23: mol_type = other DNA
cccatagactacgtctgccatct

SEQ ID NO: 13 - DNA, 31 bp, synthetic construct
  source 1..31: mol_type = other DNA
acgttggatgaaggatttgggaagcaggaac

SEQ ID NO: 14 - DNA, 29 bp, synthetic construct
  source 1..29: mol_type = other DNA
acgttggatgtgcaccggggaggaggaga

SEQ ID NO: 15 - DNA, 19 bp, synthetic construct
  source 1..19: mol_type = other DNA
caggaaccgagggaaatgc

SEQ ID NO: 16 - DNA, 30 bp, synthetic construct
  source 1..30: mol_type = other DNA
acgttggatgcctggaggcaatcaaagatg

SEQ ID NO: 17 - DNA, 30 bp, synthetic construct
  source 1..30: mol_type = other DNA
acgttggatgcttaagtcgccttggtacac

SEQ ID NO: 18 - DNA, 20 bp, synthetic construct
  source 1..20: mol_type = other DNA
caggagttaagggagaaaat

SEQ ID NO: 19 - DNA, 30 bp, synthetic construct
  source 1..30: mol_type = other DNA
acgttggatgagcctgatgcagtcacatcc

SEQ ID NO: 20 - DNA, 30 bp, synthetic construct
  source 1..30: mol_type = other DNA
acgttggatggtgtgtgccagacaaaactc

SEQ ID NO: 21 - DNA, 17 bp, synthetic construct
  source 1..17: mol_type = other DNA
gctgcctgcccagaact

SEQ ID NO: 22 - DNA, 30 bp, synthetic construct
  source 1..30: mol_type = other DNA
acgttggatgtcacattggcagggtccaac

SEQ ID NO: 23 - DNA, 30 bp, synthetic construct
  source 1..30: mol_type = other DNA
acgttggatgttatagtctgctctggactg

SEQ ID NO: 24 - DNA, 22 bp, synthetic construct
  source 1..22: mol_type = other DNA
gtcagggtccaactcagtctcc

SEQ ID NO: 25 - DNA, 30 bp, synthetic construct
  source 1..30: mol_type = other DNA
acgttggatgacaaagaacacatcgcaagg

SEQ ID NO: 26 - DNA, 30 bp, synthetic construct
  source 1..30: mol_type = other DNA
acgttggatgggcagcagctttcagcaaac

SEQ ID NO: 27 - DNA, 23 bp, synthetic construct
  source 1..23: mol_type = other DNA
caataatgtttaacgttagactc

SEQ ID NO: 28 - DNA, 30 bp, synthetic construct
  source 1..30: mol_type = other DNA
acgttggatgttctgcaacgctcgaaatcc

SEQ ID NO: 29 - DNA, 30 bp, synthetic construct
  source 1..30: mol_type = other DNA
acgttggatgtttaacctacgcatgcctcc

SEQ ID NO: 30 - DNA, 17 bp, synthetic construct
  source 1..17: mol_type = other DNA
ccaaagacctgttgaga

SEQ ID NO: 31 - DNA, 30 bp, synthetic construct
  source 1..30: mol_type = other DNA
acgttggatggtggaagaagacatcactgg

SEQ ID NO: 32 - DNA, 30 bp, synthetic construct
  source 1..30: mol_type = other DNA
acgttggatggcaggagaaaaaagcataag

SEQ ID NO: 33 - DNA, 27 bp, synthetic construct
  source 1..27: mol_type = other DNA
tcgatcttacttttttatcttccatta